(19) United States
(12) Patent Application Publication
(10) Pub. No.: US 2006/0099211 A1
(43) Pub. Date: May 11, 2006

(54) Safer, more potent human immunoglobulin preparations for treating Alzheimer's disease

(76) Inventors: Carmen Monthe, Bronx, NY (US); Paul Szabo, Linden, NJ (US); Mark Weksler, Tenafly, NJ (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 11/104,300

(22) Filed: Apr. 12, 2005

Related U.S. Application Data
(60) Provisional application No. 60/561,423, filed on Apr. 12, 2004.

(51) Int. Cl. A61K 39/395; C07K 16/18

(52) U.S. Cl. 424/145.1; 530/388.25

(57) ABSTRACT

The invention provides anti-amyloid-β immunoglobulin preparations that have been treated to remove amyloid-β ligands.

This application claims priority from U.S. Provisional Application Ser. No. 60/561,423 filed Apr. 12, 2004, which is hereby incorporated by reference in its entirety.

### FIELD OF THE INVENTION

The invention relates to improved human immunoglobulin preparations that are depleted of amyloid-β peptides and have utility for treating Alzheimer's disease.

### BACKGROUND OF THE INVENTION

Alzheimer's disease is characterized by the accumulation of plaque in brain tissues that is composed of amyloid-β (Aβ) peptides. Administration of anti-amyloid-β antibodies to a mouse model of Alzheimer's disease appears to reduce amyloid-β peptide deposits in the mouse brain and increase cognitive function. See Bard et al. (2000) Nature Medicine 6: 916-19; Bard et al. (2003) Proc. Nat'l. Acad. Sci. 100: 2023-28.

Naturally occurring antibodies against amyloid-β peptides are present in human serum, pooled human serum and in immunoglobulin preparations prepared from pooled human plasmas. Infusion of human immunoglobulin preparations into humans has been reported to be associated with a decrease in amyloid-β peptide levels in the cerebrospinal fluid. Dodel et al. (2004) J. Neurol. Neurosurg. Psychiatry 75: 1472-74; Kountouris et al. (1999) The Efficiency of Intravenous Immunoglobulin by Dementia Alzheimer Type Patients. 9^{th} Congress of the International Psychogeriatric Association; Depboylu, C., Dodel, R. C., Hampel, H., Frölich, L., Haag, A., Bürger, K., Hemmeter, U., Möller, H. J, Sommer, N., Oertel, W. H., Klockgether, T., Farlow, M., Du, Y. (2003) Human Immunoglobulins for the Treatment of Alzheimer's disease, German Society for Neurology. However, the inventors have discovered that such intravenous immunoglobulin preparations contain significant amounts of neurotoxic amyloid-β peptides and exhibit significant variation in antibody affinity and titer. Hence, procedures are needed for preparing anti-amyloid-β antibody preparations that consistently exhibit high affinities and high titers for amyloid-β peptides.

### SUMMARY OF THE INVENTION

According to the invention, immunoglobulins obtained from humans are contaminated with amyloid-β peptides that may adversely affect patients with Alzheimer's disease. Thus, the safety and efficacy of immunoglobulin preparations can be enhanced by dissociating and removing ligands such as amyloid-β peptides that are associated with such immunoglobulins. Furthermore, removal of amyloid-β peptides results in an increase in the potency of the immunoglobulin preparation, as evidenced by an increased affinity and titer of anti-amyloid-β antibodies in the immunoglobulin preparations. Because the antibodies that had bound such amyloid-β peptides were likely the antibodies with the greatest avidity for amyloid-β, the purification procedures of the invention free up the most-avid anti-amyloid-β antibodies and make them available for reaction with, and sequestration of, amyloid-β peptides after administration to patients.

Hence, the invention provides methods for removing amyloid-β ligands from immunoglobulin preparations. The method involves mixing an anti-amyloid-β immunoglobulin preparation with a protein dissociating agent, and separating immunoglobulins from amyloid-β ligands to generate an anti-amyloid-β immunoglobulin preparation that is substantially free of amyloid-β ligands.

The invention also provides anti-amyloid-β immunoglobulin preparations that have been subjected to the methods of the invention. Such immunoglobulin preparations can be naturally produced immunoglobulin preparations that are obtained from an animal, including a human, that are then treated according to the invention. Commercially available human immunoglobulin preparations can also be treated according to the methods described herein.

The immunoglobulin preparations of the invention with amyloid-β ligands removed can be used in compositions and methods for treating Alzheimer's disease.

### DESCRIPTION OF THE FIGURES

FIGS. 1A and 1B illustrate that the anti-amyloid-β (1-42) antibody avidities of different human serum preparations increased after acid treatment, but not after treatment with PBS. FIG. 1A illustrates that after treatment with PBS, human sera have a wide range of free antibody avidities for the amyloid-β (1-42) peptide. The “JD” serum had the lowest avidity. FIG. 1B illustrates that after acid treatment, all human sera had similar avidity. According to the invention, substantially all the high affinity anti-amyloid-β antibodies became accessible after acid treatment because bound amyloid-β ligands were removed.

FIGS. 2A and 2B illustrate that the anti-amyloid-β (1-42) antibody avidities of commercially available intravenous immunoglobulin preparations increased after acid treatment, but not after treatment with PBS. FIG. 2B illustrates that after treatment with acid, the 50% inflection point of these immunoglobulin preparations is at a higher value than when the immunoglobulin preparations are treated with PBS (FIG. 2A). These results indicate that the apparent titer of the immunoglobulin preparations, as measured by titration with the amyloid-β (1-42) peptide used in this assay increases after acid treatment. This shift in apparent titer most likely occurs because acid treatment dissociates bound amyloid-β ligands, thereby freeing the immunoglobulins to bind the amyloid-β (1-42) peptide.

### DETAILED DESCRIPTION OF THE INVENTION

The invention provides improved anti-amyloid-β immunoglobulin preparations and methods for making such improved immunoglobulin preparations. According to the invention, amyloid-β ligands may be bound to antibodies present in many immunoglobulin preparations and removal of such ligands provides a higher affinity and higher titer anti-amyloid-β immunoglobulin preparation. The methods of the invention involve obtaining an immunoglobulin preparation suspected of containing antibodies that can bind to amyloid-β ligands, contacting the immunoglobulin preparation with a protein dissociation agent to form a dissociation mixture and separating the antibodies from the dissociation mixture to remove amyloid-β ligands and the protein dissociation agent, thereby generating an anti-amyloid-β immunoglobulin preparation that is substantially free of ligands.

As used herein, a “ligand” is a molecule that is associated with an immunoglobulin, particularly an anti-amyloid-β immunoglobulin. Association with such a ligand can block or interfere with binding of amyloid-β peptides by the immunoglobulin, especially in the blood and spinal fluid of patients with Alzheimer's disease. While the exact sequence and structure of the amyloid-β ligands to be removed from an immunoglobulin preparation may not be known with certainty, according to the invention, the methods provided herein can remove any amyloid-β ligand from an immunoglobulin preparation. In some embodiments, the amyloid-β ligand is an amyloid-β peptide or has a structure similar to an amyloid-β peptide.

As used herein, an “immunoglobulin preparation” is a polyclonal human antibody that has been purified by the methods described herein to remove amyloid-β peptides. Such an immunoglobulin preparation can be obtained from human serum, pooled human serum, pooled human plasmas, or by other means as described herein.

The immunoglobulin preparations of the invention can bind amyloid-β peptides. Such amyloid-β peptides are derived from the amyloid-β precursor protein, which is synthesized and broken down in the brain to form amyloid-β peptides. The sequences for amyloid-β precursor proteins and variants thereof are publicly available through the website for the National Center for Biotechnology Information (http://www.ncbi.nlm.nih.gov/). One example of such an amyloid-β precursor protein sequence is provided below as SEQ ID NO: 1.

| | |
|---|---|
| 1 | MLPSLALLLL AAWTVRALEV PTDGNAGLLA EPQIAMFCGK |
| 41 | LNMHMNVQNG KWESDPSGTK TCIGTKEGIL QYCQEVYPEL |
| 81 | QITNVVEANQ PVTIQNWCKR GRKQCKTHTH IVIPYRCLVG |
| 121 | EFVSDALLVP DKCKFLHQER MDVCETHLHW HTVAKETCSE |
| 161 | KSTNLHDYGM LLPCGIDKFR GVEFVCCPLA EESDSVDSAD |
| 201 | AEEDDSDVWW GGADTDYADG GEDKVVEVAE EEEVADVEEE |
| 241 | EADDDEDVED GDEVEEEAEE PYEEATERTT STATTTTTTT |
| 281 | ESVEEVVREV CSEQAETGPC RAMISRWYFD VTEGKCVPFF |
| 321 | YGGCGGNRNN FDTEEYCMAV CGSVSTQSLL KTTSEPLPQD |
| 361 | PDKLPTTAAS TPDAVDKYLE TPGDENEHAH FQKAKERLEA |
| 401 | KHRERMSQVM REWEEAERQA KNLPKADKKA VIQHFQEKVE |
| 441 | SLEQEAANER QQLVETHMAR VEAMLNDRRR LALENYITAL |
| 481 | QAVPPRPHHV FNMLKKYVRA EQKDRQHTLK HFEHVRMVDP |
| 521 | KKAAQIRSQV MTHLRVIYER MNQSLSLLYN VPAVAEEIQD |
| 561 | EVDELLQKEQ NYSDDVLANM ISEPRISYGN DALMPSLTET |
| 601 | KTTVELLPVN GEFSLDDLQP WHPFGVDSVP ANTENEVEPV |
| 641 | DARPAADRGL TTRPGSGLTN IKTEEISEVK MDAEFGHDSG |
| 681 | FEVRHQKLVF FAEDVGSNKG AIIGLMVGGV VIATVIVITL |
| 721 | VMLKKKQYTS IHHGVVEVDA AVTPEERHLS KMQQNGYENP |
| 761 | TYKFFEQMQN |

The peptides responsible for amyloid plaque formation are typically about thirty nine to about forty three amino acids in length. Such amyloid-β peptides are neurotoxic and are implicated in the development of Alzheimer's disease. For example, the sequence of such an amyloid-β peptide (1-42) ligand is provide below (SEQ ID NO:2).

DAEFRHDSGY EVHHQKLVFF AEDVGSNKGA IIGLMVGGVV IA

In some embodiments, the immunoglobulin preparations of the invention can bind the amyloid-β peptide (1-42). In other embodiments, the immunoglobulin samples can bind the amyloid-β peptide (1-40). The sequence of an example of such an amyloid-β peptide (1-40) is provided below (SEQ ID NO:3).

DAEFRHDSGY EVHHQKLVFF AEDVGSNKGA IIGLXVGGVV

In addition to the amyloid-β peptides (1-40) and (1-42), some breakdown products of these peptides can also be detected in human blood and/or tissue samples.

As shown herein, incubation of immunoglobulin samples with pH 2.5 glycine buffer permitted removal of potentially neurotoxic amyloid-β ligands from those immunoglobulin samples and at the same time increased the avidity and apparent titer of antibodies in those preparations for amyloid-β peptides. Thus, treatment of such immunoglobulin preparations to increase the levels of free antibodies and to decrease the levels amyloid-β ligands provides an immunoglobulin preparation that is less neurotoxic and more potent for the treatment of Alzheimer's disease. Use of the present improved immunoglobulin preparations are also less likely to give rise to side effects.

While not wishing to be limited to any particular mechanism, it is believed by the inventors that the anti-amyloid-f antibodies in human serum and in commercially available immunoglobulin preparations can have bound amyloid-β ligands and may not be immediately available to bind free amyloid-β peptides in the bloodstream and tissues of patients with Alzheimer's disease. By dissociating amyloid-β ligands from these antibodies, the antibodies most capable of binding amyloid-β peptides are therefore liberated.

The immunoglobulin preparations employed in the invention are obtained from human serum or from commercially available immunoglobulin preparations. As shown previously by the inventors, human sera have antibodies against amyloid-β peptides. Weksler M. E., Relkin N., Turkenich R., LaRusse S., Zhou, L., Szabo P. (2002) Exp. Gerontol. 37(7): 943-48. Hence, either human serum or commercially available immunoglobulin preparations can be used in the practice of the invention.

The immunoglobulin preparations employed are preferably purely or antigenically human immunoglobulin. Hence, the immunoglobulin preparations of the invention are generally obtained from human sources. For example, the immunoglobulin preparation can be obtained by withdrawal of human intravenous blood. To obtain immunoglobulin preparations, individual blood or blood component samples can be pooled. Blood cells are removed to produce serum or plasma samples that are subjected to the methods described herein to generate the immunoglobulin preparations of the invention. Thus, the immunoglobulin preparations are “substantially free of cellular material.” The term “substantially free of cellular material” refers to immunoglobulin preparations where cells have been removed by available procedures. For example, cells can be removed by centrifugation.

The immunoglobulin samples used in the invention preferably have higher than normal titers of antibodies capable of binding to one or more amyloid-β peptides. The isolated immunoglobulin preparations can be whole immunoglobulin preparations. Thus, the isolated immunoglobulin sample employed is not restricted to any particular fraction or isotype and may be IgG, IgM, IgA, IgD, IgE, or any combination thereof.

In some embodiments, the immunoglobulin preparation may be enriched for the IgG fraction or isotype. If IgG fractions are desired, they can be prepared by available procedures.

Human antibodies can also be produced recombinantly by obtaining a nucleic acid that encodes a high affinity anti-amyloid-β antibody from an experimental animal and replacing non-human DNA sequences of this nucleic acid with human antibody DNA sequences to generate a nucleic acid that codes for a “humanized” antibody while retaining the antigen binding ability of the original antibody molecule. Procedures available in the art can be used for these manipulations.

In general, any available protein dissociation agent can be used to free the anti-amyloid-β immunoglobulins from associated ligands. Solutions of protein dissociation agents that can be used include acidic solutions, urea solutions, guanidinium hydrochloride solutions, detergent solutions, high salt solutions and the like. For example, an acidic solution having a pH of about 2.0 to about 4.0, or a pH of about 2.5 to about 4.0, or a pH of about 2.0 to about 3.0, or a pH of about 2.5 to about 3.0, can be used to dissociate anti-amyloid-β immunoglobulins from associated ligands. Alternatively, a urea solution having about 2 molar urea to about 8 molar urea can be used to dissociate anti-amyloid-β immunoglobulins from associated ligands.

Once the immunoglobulins and amyloid-β ligands have been dissociated, a variety of techniques can be used to separate the liberated immunoglobulins from the amyloid-β ligands. These include purification methods based on the size or molecular weight difference of human immunoglobulins (for example, about 160 kD or larger) and most amyloid-β ligands (about 4 kD). Other separation methods that can be employed include separation methods based on charge, hydrophobicity, and affinity. Methods such as molecular sieve filtration, centrifugation, dialysis, ion-exchange chromatography, ammonium sulfate differential precipitation, affinity column chromatography can be used.

Thus, for example, the smaller molecular weight amyloid-β ligands can be removed from the immunoglobulin preparation by filtration through a commercially available protein concentration filter, for example, an Amicon or Millipore Pellicon ultrafiltration unit. Filter pore sizes are selected to allow passage of the desired range of molecular weights, for example, about 0.5 to about 20 kilodaltons, or about 1 to about 10 kilodaltons, for many of the amyloid-β peptides. Centrifugation or pressure can be used to facilitate passage of the smaller molecular weight amyloid-β ligands through the filter. Similarly, the liberated inmmunoglobulins can be separated from the amyloid-β ligands by dialysis, where the dialysis tubing permits the smaller molecular weight amyloid-β ligands to pass through the tubing, but where the larger immunoglobulin molecules are retained within the dialysis bag. Dialysis can also be used to remove the protein dissociating agent. However, removal of the protein dissociating agent should generally be done after removal of the amyloid-β ligands. Hence, in many embodiments, it may be preferable to retain the protein dissociating agent in the immunoglobulin sample while removing the amyloid-β ligands.

Alternatively, or in addition to this filtration step, the immunoglobulin preparations can be applied to a purification matrix such as a gel filtration medium that will also allow separation of the immunoglobulins from the amyloid-β ligands on the basis of molecular size. Anion or cation exchange resin can be employed for separation of immunoglobulins and amyloid-β ligands. For example, a matrix or substrate having pendant diethylaminoethyl (DEAE) groups can be used. Other separation matrices that can be employed include acrylamide, agarose, dextran, cellulose or other types of matrixes commonly employed in protein purification.

As used herein, the immunoglobulins are said to be “isolated” or “purified” when they are substantially free of amyloid-β ligands, cellular material and protein dissociating agents and/or other chemicals or antigens that may cause adverse side effects in vivo. The immunoglobulins of the present invention can be purified to homogeneity or other degrees of purity. However, in some embodiments, the level of purification need only be sufficient to separate the immunoglobulins from amyloid-β ligands and from undesirable chemicals such as the protein dissociation agent(s) used.

Thus, the immunoglobulin preparations of the invention can have less than about 30% (by dry weight) other proteins (i.e., contaminating protein), less than about 20% other proteins, less than about 10% other proteins, or less than about 5% other proteins, or less than about 3% other proteins.

After separation of free anti-amyloid-β immunoglobulins from amyloid-β ligands, the protein dissociating agent is removed from the anti-β-amyloid immunoglobulin preparations, for example, by a combination of dialysis, gel filtration, ultra-filtration or other means.

Affinity purification of an antibody pool or sera may also provide a practitioner with a more uniform reagent. For example, smaller amounts of immunoglobulin preparations may be administered after those preparations have been affinity purified to remove non-amyloid-β antibodies. Methods for enriching anti-amyloid β antibodies using antibody affinity matrices to form an affinity column are available in the art. Briefly, amyloid-β peptides can be attached to an affinity support (e.g., CNBR Sepharose™, Pharmacia Biotech). An immunoglobulin preparation is then passed over the affinity matrix and the amyloid-β antibodies bind. Unbound antibodies are washed off the matrix. Bound antibodies are released by techniques available to those familiar with the art, yielding a concentrated immunoglobulin preparation that is substantially free of non-anti-amyloid-β antibodies. This enriched anti-amyloid-β antibody pool can then be used in the compositions and methods of the invention.

The immunoglobulin preparations of the invention can be concentrated adjust the concentration of antibodies in the preparation. For example, the immunoglobulin preparations can be concentrated by ultrafiltration.

Typically, the immunoglobulin preparations are placed in pharmaceutically acceptable carriers or solutions before administration. For example, after removal of amyloid-β peptides, and other contaminants if desired, the free anti-amyloid-β immunoglobulins can be dialyzed against physiologically compatible solutions. Pharmaceutical compositions containing the immunoglobulin preparations of the invention can be packaged in unit doses. Suitable carriers include glycine or saline solutions as well as other materials commonly used in the art.

The immunoglobulin preparations can be formulated for parenteral administration (e.g., by injection, for example, bolus injection or continuous infusion) and may be presented in unit dosage form in ampoules, prefilled syringes, small volume infusion containers or multi-dose containers with an added preservative. Such pharmaceutical compositions typically are formulated as solutions, and occasionally as suspensions. In some instances it may be useful to include formulatory agents such as suspending, stabilizing and/or dispersing agents in the immunoglobulin compositions.

Therapeutic Methods

The immunoglobulin preparations of the invention can be administered intravenously or subcutaneously to patients to prevent or treat Alzheimer's disease. The methods described herein for freeing immunoglobulin preparations from associated ligands increase the level of free anti-amyloid-β antibodies and decrease the level of amyloid-β peptides. Such ligand-free immunoglobulin preparations enhance the therapeutic efficacy of anti-amyloid-β immunoglobulin preparations and reduce risk of toxicity during intravenous immunoglobulin treatment in patients with Alzheimer's disease. Use of the present immunoglobulin preparations therefore has significant advantages over currently available immunoglobulin preparations in the treatment of Alzheimer's patients.

Administration of immunoglobulin preparations may take place in a single dose or in a dose repeated once or several times over a certain period. The appropriate dosage varies according to various parameters. Such parameters include the physiological status of the individual treated, the immunoglobulin preparation, the mode and frequency of administration, and the like.

Dosages of the immunoglobulin preparations of the invention can vary depending upon the age, weight and physiological status of the patient. In some embodiments, the immunological preparations of the invention are administered once a week or once every two weeks. The amount of immunoglobulin administered can vary and may depend upon the purity, affinity and avidity of the immunoglobulin preparation. For example, about 0.01 g per kg body weight to about 5 g per kg body weight, or about 0.05 g per kg body weight to about 3 g per kg body weight, or about 0.1 g per kg body weight to about 2 g per kg body weight of immunoglobulin can be administered once a week or once every two weeks.

Methods for administration of immunoglobulins are available in the art. For example, intravenous administration of immunoglobulins is commonly used in the treatment of immunodeficient patients, patients with idiopathic thrombocytopenic purpura, and patients with a variety of autoimmune diseases. Immunoglobulin preparations are also commonly administered subcutaneously.

Kits for treating or preventing Alzheimer's disease in a patient are also provided. These kits can include an immunoglobulin preparation of the invention. Other components can also be provided in the kits of the invention. For example, the kit can include a means for administering the immunoglobulin preparation to a patient, such as a syringe.

The invention is further illustrated by the following non-limiting Examples.

### EXAMPLE 1

### Currently Available Immunoglobulin Preparations Contain Amyloid-β Peptides

This Example shows that human intravenous immunoglobulin (IVIg) preparations contain neurotoxic amyloid-β peptides.

Wild type C57BL/6 mice were used for this study. Prior to infusion of IVIg), no human amyloid-β peptides were detected in the sera of these mice. However, 24 hours after intraperitoneal administration of 1.2 gm/kg human IVIg, human amyloid-β peptides were detected in the blood of these mice using a sandwich ELISA employing an anti-N-terminal amyloid-β peptide antibody, which discriminates human from mouse amyloid-β peptides. Serum from mice contained human amyloid-β peptides at concentration of 220-320 pg/ml. As the ELISA is specific for human Aβ peptides, the source of the human Amyloid-β peptides could only have been the injected IVIg. Subsequently it was determined that the concentration of Amyloid-β peptide in a comparable lot of IVIg was approximately 900 pg/ml.

Thus, human IVIg can contain about 900 pg of Amyloid-β peptides per 10 mg of IVIg. For this reason, infusion of currently available IVIg preparations may increase the level of neurotoxic Aβ peptides in the blood of recipients and, therefore, be a risk for patients with Alzheimer's disease.

### EXAMPLE 2

### Removal of Ligands from Immunoglobulin Preparations Improves the Avidity and Titer of Anti-Amyloid-β Antibodies Therein

This Example illustrates that incubation of human sera and immunoglobulin samples with pH 2.5 glycine buffer, permitted removal of potentially neurotoxic amyloid-β ligands from those sera and immunoglobulin samples. Such treatment increased the apparent avidity and titer of antibodies in those sera and samples for amyloid-β peptides.

Materials and Methods

Human sera were obtained from six individuals after obtaining informed consent. Immunoglobulin preparations were obtained from BAXTER. Human sera and BAXTER immunoglobulin preparations that had been diluted 1:3 in PBS, were further diluted 1:1 in PBS as a control, or in acid buffer (PBS buffer with 1.5% BSA and 0.2M glycine-acetate pH 2.5) to dissociate the antibodies from amyloid-β ligands. After 10-15 minutes incubation at room temperature, the samples were pipetted into Millipore Ultrafree-MC centrifugal devices (10,000 MWCO) and centrifuged at 10,000×g for 20 minutes at room temperature. The level of free anti-amyloid-β antibodies in the retentate solutions were assayed after neutralization on amyloid-β (1-42) coated ELISA plates at multiple dilutions. The binding properties of the retained antibodies were assessed in two ways. First, the antibody titer for the amyloid-β (1-42) peptide was calculated as the dilution at which the absorbance was no greater than the negative control. Second, the antibody avidity for the amyloid-β (1-42) peptide was calculated as the antibody dilution at the inflection point of the curve plotting optical density versus immunoglobulin dilution.

Results

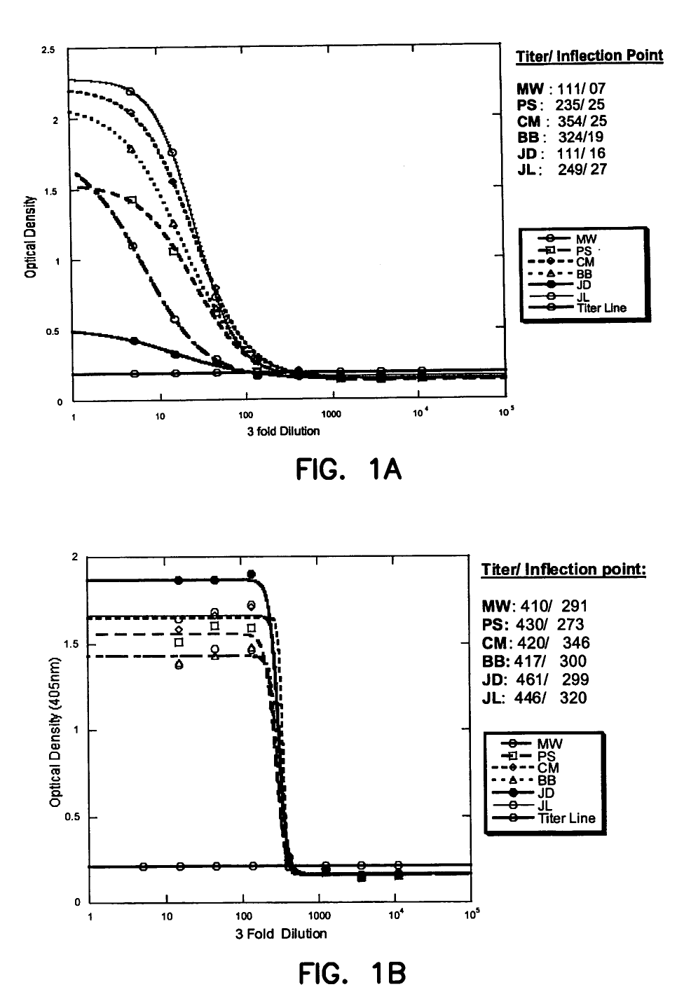

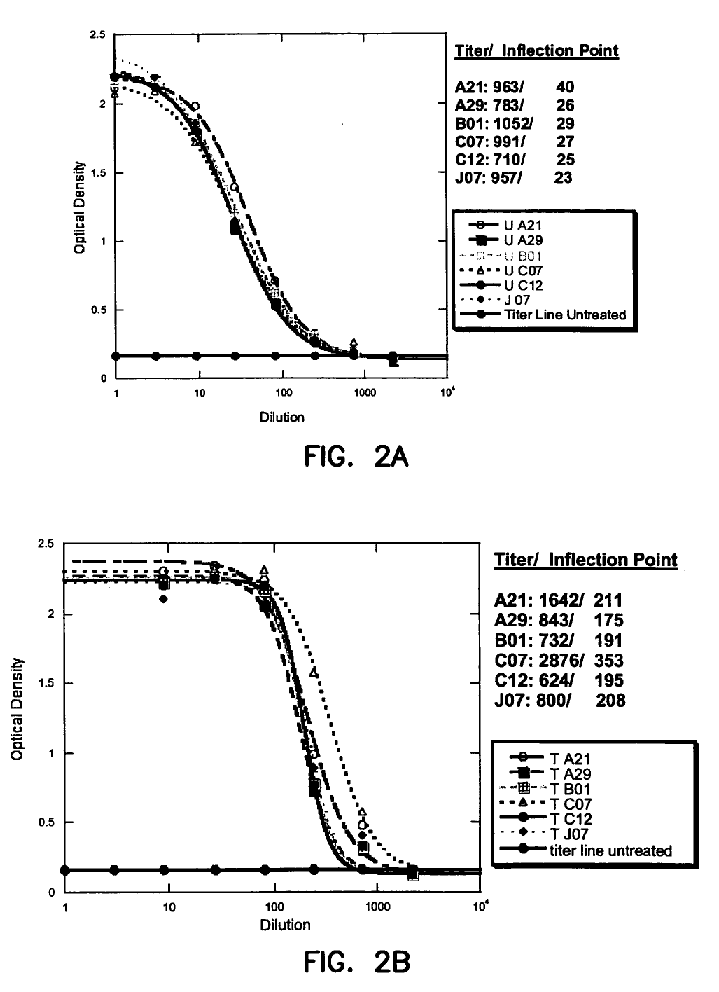

As shown in FIGS. 1A and 1B, acid-treated human sera had higher titers of free anti-amyloid antibodies than did PBS-treated human sera. Similarly, FIGS. 2A and 2B show that acid-treated immunoglobulin preparations had higher apparent titers of free anti-amyloid-β antibodies compared with PBS treated immunoglobulin preparations.

Similar results were obtained with immunoglobulin preparations from other manufacturers including Sandoglobulin, Octapharma, Aventis and Bayer. Preliminary results indicate that thiocyanate elution can also be used as a method for separating anti-amyloid-β antibody:ligand complexes that exist in immunoglobulin preparations.

These results indicate that acid treatment of immunoglobulin preparations and human sera increase the apparent antibody titer and avidity by dissociating antibody:amyloid-β complexes that exist in the immunoglobulin preparations.

### EXAMPLE 3

### Amyloid-β Levels in Alzheimer's Patients' Cerebrospinal Fluids Decrease after Treatment with Anti-Amyloid-Beta Peptide (Aβ) Antibodies

Previous studies indicate that infusion of anti-amyloid-beta peptide (Aβ) antibodies into APP-transgenic mice reverses cerebral amyloid deposits and cognitive decline. This Example illustrates that amyloid-β peptide levels are reduced after treatment with immunoglobulin preparations containing anti-amyloid-β antibodies.

A dose-range study of immunoglobulin (IVIg) containing anti-Amyloid-β antibodies was performed in patients with Alzheimer's Disease. The study was designed to last about six months and utilized eight patients with mild to moderate Alzheimer's Disease, as measured by the Mini-Mental State Examination (MMSE), a standard clinical test used to gauge general mental abilities. The mean MMSE score for these patients was 23.5, with a range of 20-29. A single lot of IVIg with elevated anti-Aβ antibody titers was employed. Two patients were randomly assigned to each of four IVIg dosing regimens (0.4 g/kg/2 weeks, 0.4 g/kg/1 week, 1 g/kg/2 weeks or 2 g/kg/month). Anti-Aβ antibodies and Aβ levels in blood and cerebrospinal fluid (CSF) before and after IVIg infusions were quantified by ELISA. MMSE were performed before and after 6 months of IVIg therapy.

After each infusion of IVIg, anti-Aβ antibody levels in plasma of every patient increased in a dose-dependent fashion. Anti-Aβ antibody levels rose progressively with successive treatments when IVIg was given once/week or once/every two weeks but not once/month. These data indicate that the anti-Aβ antibodies may have a shorter half-life, 10-13 days, compared to the reported half-life of 21-36 day of IgG in normal subjects.

The shorter half-life of anti-Aβ antibody in patients with Alzheimer's Disease is consistent with a conclusion that the infused anti-Aβ antibodies bound to endogenous Aβ. Infusion of 1 gm of IVIg/kg every 2 weeks was associated with both an increase in the level of plasma Aβ42 and a decrease in the level of Aβ42 in the CSF. These results suggest that treatment of AD patients with IVIg mobilizes Aβ from the brain into the CSF and then into bloodstream.

After infusion of 0.4 gm/kg IVIg, the serum IgG was 16-17 mg/ml and after infusion of 2 gm/kg IVIg, the serum IgG was 50 mg/ml. As values above 20 mg/ml are reported to shorten the half life of IgG, use of a highly enriched anti-amyloid-β antibody preparations would not only allow smaller infusions (more convenient for home therapy) that deliver same amount of anti-AB antibodies but potentially would prolong the half life of the anti-AB by decreasing the increase in IgG levels.

To date, MMSE scores have increased in all Alzheimer's patients who have completed 6 months of IVIg therapy. Taken together these results suggest that infusion of IVIg containing anti-Amyloid-β antibodies may be an effective treatment for Alzheimer's Disease.

### REFERENCES

(1) DeMattos, R. B., Bales, K. R., Cummins, D. J., Paul, S. M., Holtzman, D. M. (2002) Science. 295: 2264-2267

(2) Depboylu, C., Dodel, R. C., Hampel, H., Frölich, L., Haag, A., Bürger, K., Hemmeter, U., Möller, H. J, Sommer, N., Oertel, W. H., Klockgether, T., Farlow, M., Du, Y. (2003) Human Immunoglobulins for the Treatment of Alzheimer's disease. German Society for Neurology.

(3) Dodel, R., Hampel, H., Depboylu, C., Lin, S., Gao, F., Schock, S., Jäckel, S., Wei, X., Buerger, K., Höft, C., Hemmer, B., Möller, H. J., Farlow, M., Oertel, W., Sommer, N., Du, Y. (2002) Ann. Neurol. 52: 253-256

(4) Kountouris, D. (1999) The Efficiency of Intravenous Immunoglobulin by Dementia Alzheimer Type Patients. 9^{th} Congress of the International Psychogeriatric Association

(5) Kazatchkine, M. D., Kaveri, S. V. (2001) Immunomodulation of autoimmune and inflammatory diseases with intravenous immune globulin. N. Engl. J. Med. 345:747-55.

(6) Weksler M. E., Relkin N., Turkenich R., LaRusse S., Zhou, L., Szabo P. (2002) Exp. Gerontol. 37(7): 943-48.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. An isolated human polyclonal immunoglobulin preparation that can specifically bind an amyloid-β peptide; wherein the immunoglobulin preparation is substantially free of amyloid-β ligands.

2. The isolated immunoglobulin preparation of claim 1, wherein the amyloid-β peptide has SEQ ID NO:2 or SEQ ID NO:3.

3. The isolated immunoglobulin preparation of claim 1, wherein the amyloid-β ligand is a peptide derived from an amyloid-β protein having SEQ ID NO: 1.

4. The isolated immunoglobulin preparation of claim 1, wherein the isolated, purified immunoglobulin preparation has been treated with a protein dissociating reagent to release bound amyloid-β ligands.

5. The isolated immunoglobulin preparation of claim 4, wherein the protein dissociating reagent is an acidic solution.

6. The isolated immunoglobulin preparation of claim 5, wherein the acidic solution is an aqueous solution of about pH 2.0 to about pH 4.0.

7. The isolated immunoglobulin preparation of claim 5, wherein the acidic solution is an aqueous solution of about pH 2.0 to about pH 3.0.

8. The isolated immunoglobulin preparation of claim 4, wherein the protein dissociating reagent is a urea solution.

9. The isolated immunoglobulin preparation of claim 8, wherein the urea solution is a solution of about 2 molar to about 8 molar.

10. The isolated immunoglobulin preparation of claim 4, wherein antibodies in the immunoglobulin preparation have been separated from amyloid-β ligands after treatment with the protein dissociating reagent.

11. The isolated immunoglobulin preparation of claim 10, wherein the antibodies have been separated from amyloid-β ligands by filtration, molecular sieve filtration, centrifugation, dialysis, ion-exchange chromatography, ammonium sulfate differential precipitation, or affinity column chromatography.

12. The isolated immunoglobulin preparation of claim 1, which is substantially free of non-anti-amyloid-β antibodies.

13. A composition comprising a pharmaceutically acceptable carrier and an effective amount of the isolated immunoglobulin preparation of claim 1.

14. A kit comprising an isolated immunoglobulin preparation that can bind an amyloid-β peptide and instruction for using the immunoglobulin preparation for treating Alzheimer's disease; wherein the isolated immunoglobulin preparation is substantially free of amyloid-β ligands.

15. A method of preparing an anti-amyloid-β immunoglobulin preparation that is substantially free of amyloid-β ligands comprising: (a) obtaining an immunoglobulin preparation that contains antibodies that can bind an amyloid-β peptide; (b) mixing the immunoglobulin preparation with a protein dissociating reagent to release amyloid-β ligands from antibodies in the immunoglobulin preparation; and (c) separating antibodies in the immuno-globulin preparation from amyloid-β ligands to generate an anti-amyloid-β immunoglobulin preparation that is substantially free of amyloid-β ligands.

16. The method of claim 15, wherein the protein dissociating reagent is an acidic solution.

17. The method of claim 16, wherein the acidic solution is an aqueous solution of about pH 2.0 to about pH 4.0.

18. The method of claim 16, wherein the acidic solution is an aqueous solution of about pH 2.5 to about pH 3.0.

19. The method of claim 15, wherein the protein dissociating reagent is a urea solution.

20. The method of claim 15, wherein antibodies in the isolated, purified immunoglobulin preparation are separated from amyloid-β ligands by filtration, molecular sieve filtration, centrifugation, dialysis, ion-exchange chromatography, ammonium sulfate differential precipitation, or affinity column chromatography.

21. A method of treating or preventing Alzheimer's disease in a patient comprising administering to the patient a therapeutically effective amount of an anti-amyloid-β immunoglobulin composition that is substantially free of amyloid-β ligands.


SEQUENCE LISTING (3 sequences)

SEQ ID NO: 1 - protein, 770 aa, Homo sapiens
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVREVCSEQAETGPCRAMISRWYFDVTEGKCVPFFYGGCGGNRNNFDTEEYCMAVCGSVSTQSLLKTTSEPLPQDPDKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 42 aa, Homo sapiens [sequence omitted]

SEQ ID NO: 3 - protein, 40 aa, Homo sapiens
  SITE 35: Xaa = any amino acid
DAEFRHDSGYEVHHQKLVFFAEDVGSNKGAIIGLXVGGVV